(19) United States
(12) Patent Application Publication
(10) Pub. No.: US 2021/0241922 A1
(43) Pub. Date: Aug. 5, 2021

(54) METHOD AND DEVICE FOR IDENTIFYING CLUES ABOUT MEDICAL ADVERSE EVENTS, ELECTRONIC EQUIPMENT, AND MEMORY MEDIUM

(71) Applicant: Winning Health Technology Group Co., Ltd., Shanghai (CN)

(72) Inventors: Chun FAN, Shanghai (CN); Ya LI, Shanghai (CN); Ning LIU, Shanghai (CN); Xiaobo WANG, Shanghai (CN); Jiaojiao HAN, Shanghai (CN); Anqi XU, Shanghai (CN); Tao WANG, Shanghai (CA); Wei ZHOU, Shanghai (CN)

(73) Assignee: Winning Health Technology Group Co., Ltd., Shanghai (CN)

(21) Appl. No.: 16/986,702

(22) Filed: Aug. 6, 2020

(30) Foreign Application Priority Data
Feb. 3, 2020 (CN) 202010079364.9

(51) Int. Cl. G16H 50/70; G06F 16/245; G16H 10/60; G16H 70/20; G16H 70/60; G16H 15/00; G16H 40/20

(52) U.S. Cl. CPC G16H 50/70; G06F 16/245; G16H 10/60; G16H 40/20; G16H 70/60; G16H 15/00; G16H 70/20

(57) ABSTRACT

The present disclosure provides a method and a device for identifying clues about medical adverse events, electronic equipment, and a memory medium. This method includes the steps of: acquiring diagnosis and treatment information on a patient; acquiring a knowledge base of clinical pathway medical orders or a keyword library of medical adverse events corresponding to the diagnosis and treatment information; and determining whether a clue about medical adverse events exists in the diagnosis and treatment information based on the knowledge base of clinical pathway medical orders or the keyword library of medical adverse events.

### CROSS-REFERENCE TO RELATED REFERENCES

The present application claims priority of Chinese Patent Application No. 202010079364.9, filed with the Chinese Patent Office on Feb. 3, 2020 and entitled “Method and Device for Identifying Clues about Medical Adverse Events, Electronic Equipment, and Memory Medium”, the contents of which are incorporated herein by reference in their entirety.

### TECHNICAL FIELD

The present disclosure relates to the technical field of data processing, and particularly to a method and a device for identifying clues about medical adverse events, an electronic equipment, and a memory medium.

### BACKGROUND

Medical adverse events refer to unexpected, undesired or potentially dangerous events or errors that occur during the diagnosis and treatment process of patients. In the course of medical adverse events, clues about medical adverse events are formed. By identifying clues about medical adverse events, medical adverse events can be discovered.

At present, the manner of identifying clues about medical adverse events is manual identification, that is, to identify whether a clue about a medical adverse event exists by manually searching whether an inappropriate medical action in the diagnosis and treatment information on a patient exists. However, this manner requires to consume a lot of human resource and time, resulting in relatively great labor costs and low efficiency.

### SUMMARY

An object of the embodiments of the present disclosure is to provide a method and a device for identifying clues about medical adverse events, an electronic equipment, and a memory medium.

An embodiment of the present disclosure provides a method for identifying clues about medical adverse events, the method comprising the following steps of:

acquiring diagnosis and treatment information on a patient;

acquiring a knowledge base of clinical pathway medical orders or a keyword library of medical adverse events which is corresponding to the diagnosis and treatment information; and

determining, based on the knowledge base of clinical pathway medical orders or the keyword library of medical adverse events, whether a clue about medical adverse events exists in the diagnosis and treatment information.

An embodiment of the present disclosure further provides a device for identifying a clue about medical adverse events, the device comprising:

an acquisition module for diagnosis and treatment information configured to acquire diagnosis and treatment information on a patient;

an acquisition module for other information configured to acquire a knowledge base of clinical pathway medical orders or a keyword library of medical adverse events which is corresponding to the diagnosis and treatment information; and

an identification module configured to determine whether a clue about medical adverse events exists in the diagnosis and treatment information based on the knowledge base of clinical pathway medical orders or the keyword library of medical adverse events.

An embodiment of the present disclosure provides an electronic equipment, comprising a processor and a memory in which a computer readable instruction is stored, wherein the steps in the method provided above are implemented when the computer readable instruction is executed by the processor.

Other features and advantages of the present disclosure will be described in the subsequent description, and a part thereof becomes obvious from the description or would be understood by implementing the embodiments of the present disclosure. The objects and other advantages of the present disclosure will be realized and obtained through a structure specifically indicated in the written description, claims and drawings.

### BRIEF DESCRIPTION OF THE DRAWINGS

In order to more clearly describe the technical solutions of the embodiments of the present disclosure, the figures required to be used in the embodiments of the present disclosure will be simply presented below; and it shall be understood that the following figures merely show certain embodiments of the present disclosure, and thus should not be construed as limitations on the scope thereof, and for a person ordinarily skilled in the art, further relevant figures could be obtained according to these figures without inventive efforts.

FIG. 1 is a structural schematic diagram of an electronic equipment for implementing a method for identifying clues about medical adverse events provided in an embodiment of the present disclosure;

FIG. 2 is a flow chart of a method for identifying clues about medical adverse events provided in an embodiment of the present disclosure; and

FIG. 3 is a structure diagram of a device for identifying clues about medical adverse events provided in an embodiment of the present disclosure.

### DETAILED DESCRIPTION OF THE EMBODIMENTS

The technical solutions in the embodiments of the present disclosure will be clearly and comprehensively described below with reference to the figures in the embodiments of the present disclosure.

An embodiment of the present disclosure provides a method for identifying clues about medical adverse events, so as to make improvements regarding the problems in the prior art that the identification of medical adverse events requires great human resource consumption and time consumption, resulting in relatively great labor costs and low efficiency, in which whether a clue about medical adverse events exists in the diagnosis and treatment information can be determined based on the knowledge base of clinical pathway medical orders or the keyword library of medical adverse events verify on the basis of other supplementary information the situation whether a clue about medical adverse events exists during the diagnosis and treatment process, by determining knowledge base of clinical pathway medical orders keyword library of medical adverse events, accordingly, it is possible to quickly discover a clue about medical adverse events, and by initiatively digging out clues about medical adverse events, manual intervention in the identification of medical adverse events is reduced, manpower consumption is lowered, and the work efficiency is improved.

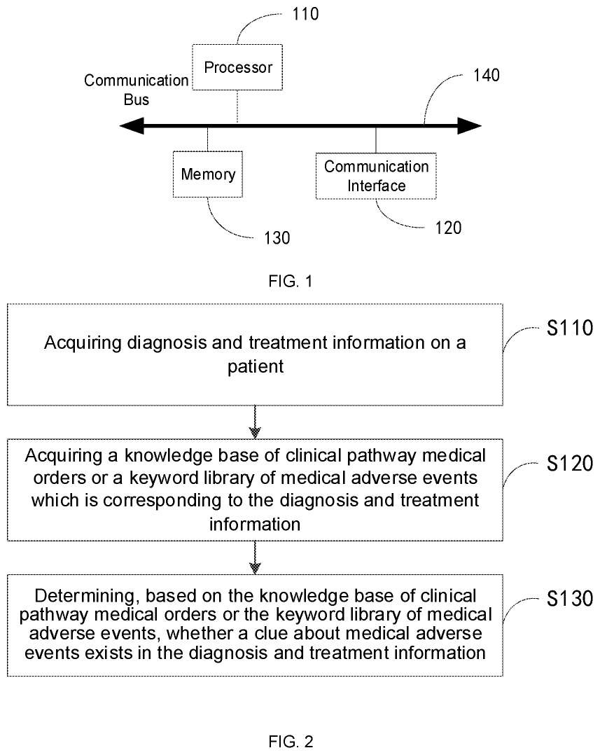

Referring to FIG. 1, FIG. 1 is a structural schematic diagram of an electronic equipment for implementing a method for identifying clues about medical adverse events provided in an embodiment of the present disclosure, wherein the electronic equipment may comprise: at least one processor 110, which is CPU for example, at least one communication interface 120, at least one memory 130, and at least one communication bus 140. In the above, the communication bus 140 is configured to realize direct connection and communication between these components. The communication interface 120 of the apparatus in the embodiment of the present disclosure is configured to perform signaling or data communication with other node apparatuses. The memory 130 may be a high-speed RAM (Random Access Memory), or a non-volatile memory, e.g. at least one magnetic-disk memory. Optionally, the memory 130 may further be at least one memory device remotely provided from the foregoing processor. A computer readable instruction is stored in the memory 130, and when the computer readable instruction is executed by the processor 110, the electronic equipment implements the method process as shown in FIG. 2 below, for example, the memory 130 may be configured to store diagnosis and treatment information on patients, and the processor 110 may acquire diagnosis and treatment information from the memory 130 and analyze the diagnosis and treatment information, so as to find out whether a clue about medical adverse events exists.

It could be understood that the structure shown in FIG. 1 is merely schematic, and the electronic equipment may further comprise more or fewer components than those shown in FIG. 1, or may have a configuration differing from that shown in FIG. 1. The components shown in FIG. 1 may be implemented by hardware, software or a combination thereof.

Referring to FIG. 2, FIG. 2 is a flow chart of a method for identifying clues about medical adverse events provided in an embodiment of the present disclosure, the method comprising the following steps of:

Step S110: acquiring diagnosis and treatment information on a patient.

In case that the medical personnel conduct improper diagnosis and treatment actions, that is to say, a clue about a medical adverse event is generated, it would be hidden in the diagnosis and treatment information, and the clue about the medical adverse event can be dug out by screening the diagnosis and treatment information, and improper diagnosis and treatment actions can be intervened timely. Real-time analysis of the diagnosis and treatment information recorded by the medical care personnel is required, if the tracing of clues about medical adverse events during the diagnosis and treatment process of patients is to be realized.

The diagnosis and treatment information refers to records of medical services made by medical care personnel, e.g. electronic records of medical orders, records of course of disease, nursing records and the like, thus, diagnosis and treatment information on the patient can be acquired from the database storing these electronic records.

The diagnosis and treatment information may contain diagnosis and treatment information of the patient corresponding to respective diagnosis and treatment stages during a process of the diagnosis and treatment, and the diagnosis and treatment information is to be analyzed every time after that the medical care personnel complete the logging (input) and storage of the diagnosis and treatment information.

The patient may refer to any random patient, and may refer to one or more patients; when more patients are involved, for each patient, the diagnosis and treatment information of the patient shall be analyzed according to the method provided in an embodiment of the present disclosure, and whether a clue about medical adverse events exists in the diagnosis and treatment information is accordingly analyzed.

Step S120: acquiring a knowledge base of clinical pathway medical orders or a keyword library of medical adverse events which is corresponding to the diagnosis and treatment information.

The clinical pathway refers to a programmed and standardized diagnosis and treatment technology having strict requirements on working sequences and time, which is established by the medical care personnel on the basis of the evidence-based medicine targeting at certain disease entities, in other words, for certain disease entities, the clinical pathway thereof prescribes that respective examinations shall be performed in a certain sequence and diagnosis and treatment measures shall be made according to the examination results, that is to say, the clinical pathway makes a specific schedule on time, procedure as well as diagnosis and treatment items of the diagnosis and treatment of patients.

Therefore, the diagnosis and treatment information on a patient can be analyzed, so as to know the disease of the patent and then acquire the clinical pathway corresponding to the disease. It could be understood that clinical pathways corresponding to various disease entities may be stored in the database, the disease of the patient is recorded in the diagnosis and treatment information of the patient, thus, the disease of the patient can be directly extracted from the diagnosis and treatment information, and then the clinical pathway corresponding to the disease can be obtained just by searching in the database.

The knowledge base of clinical pathway medical orders refers to various medical orders prescribed for the clinical pathways, and the keyword library of medical adverse events refers to certain keywords previously set for medical adverse events, and it could be understood that for each certain disease, corresponding knowledge base of clinical pathway medical orders and keyword library of medical adverse events can be preset, or for all diseases, unified knowledge base of clinical pathway medical orders and keyword library of medical adverse events can be preset; in this way, the knowledge base of clinical pathway medical orders and the keyword library of medical adverse events corresponding to the diagnosis and treatment information can be obtained.

Step S130: determining, based on the knowledge base of clinical pathway medical orders or the keyword library of medical adverse events, whether a clue about medical adverse events exists in the diagnosis and treatment information.

Since relevant medical order contents and keywords on medical adverse event and the like are prescribed in the knowledge base of clinical pathway medical orders or in the keyword library of medical adverse events, on the basis of the knowledge base of clinical pathway medical orders or the keyword library of medical adverse events, it can be determined whether a clue about medical adverse events exists in the diagnosis and treatment information, for example, relevant contents in the knowledge base of clinical pathway medical orders or in the keyword library of medical adverse events can be matched with the diagnosis and treatment information, wherein it can be determined that a clue about medical adverse events exists in the diagnosis and treatment information if the issued medical order exceeds the scope or a matched keyword is discovered.

In the above implementation process, clues about medical adverse events can be dug out initiatively by determining whether a clue about medical adverse events exists in the diagnosis and treatment information based on the knowledge base of clinical pathway medical orders or the keyword library of medical adverse events, which reduces manual intervention in the identification of medical adverse events, lowers labor consumption, and improves work efficiency.

Here, in order to facilitate analysis of the diagnosis and treatment information, the diagnosis and treatment information may be stored in the ways shown as follows in Table 1.

TABLE 1

| diagnosis and treatment actions | diagnosis and treatment information |
|---|---|
| medical orders issued by doctors | medicine-related medical orders |
| | examination-related medical orders |
| | test-related medical orders |
| | operation-related medical orders |
| | treatment-related medical orders |
| | material-related medical orders |
| | advisement-related medical orders |
| | blood transfusion-related medical |
| | orders |
| | other medical orders |
| case history written by doctors | admission records |
| | initial records of course of disease |
| | daily records of course of disease |
| | records of ward rounds of superiors |
| | records of discussion on |
| | interactable cases |
| | shift records |
| | consultation notes |
| | transfer records |
| | phase summary |
| | emergency treatment records |
| | preoperative discussion records |
| | preoperative summary |
| | anesthesia records |
| | operation notes |
| | postoperative initial records of |
| | course of disease |
| | death records |
| | records of death case discussion |
| | surgical safety check records |
| | discharge records |
| nursing history written by nurses | initial nursing records |
| | general nursing record |
| | special nursing record |
| | shift records |
| | admission nursing valuation list |
| | first-level nursing records |
| | critically ill patient valuation list |
| | reporting and determinative table of |
| | bedsore situations |
| | fall assessment |
| | catheter assessment |

As an embodiment, after the diagnosis and treatment information in the above forms is obtained, in the process of determining whether a clue about medical adverse events exists in the diagnosis and treatment information, the clinical pathway corresponding to the diagnosis and treatment information can be obtained by matching the diagnosis and treatment information on a patient with the clinical pathway on the basis of the diagnosis and treatment information on the patient, and it can be determined whether a clue about medical adverse events exists in the diagnosis and treatment information by matching the knowledge base of clinical pathway medical orders or the keyword library of medical adverse events corresponding to this clinical pathway with the acquired diagnosis and treatment information.

Prevention and control can be realized from the generation process of the diagnosis and treatment information by performing the above-mentioned matching of the knowledge base of clinical pathway medical orders or the keyword library of medical adverse events, for example, the precaution and early warning of medial adverse events can be further strengthened, if clues about medical adverse events are investigated and verified as soon as medical orders are issued.

During the above implementation process, an abnormal medical action of a doctor during the diagnosis and treatment process can be discovered in time, by matching the diagnosis and treatment information on a patient with the knowledge base of clinical pathway medical orders or the keyword library of medical adverse events, accordingly, monitoring of medical adverse events can be realized in the diagnosis and treatment process of the patient.

Since not all diseases are covered by currently available clinical pathways, in order to prevent medical adverse events related to other diseases out of the clinical pathways, a keyword library of medical adverse events is preset in the system as another approach for identifying clues about medical adverse events. It could be understood that the keyword library of medical adverse events includes keywords on e.g. diagnosis of patients and disease symptoms related to medical adverse events, which may appear in medical documents of all kinds of diseases, and the knowledge base of clinical pathway medical orders is obtained form medical order contents of currently established 1212 clinical pathways. In this way, it is possible to improve the verification of medical adverse events by identifying whether a clue about medical adverse events is contained in the diagnosis and treatment information.

It could be understood that the knowledge base of clinical pathway medical orders is set for clinical pathways, thus, it can be firstly judged whether the diagnosis and treatment information conforms to the clinical pathway participation criteria, that is to say, it is judged whether the diagnosis and treatment information matches the clinical pathways. If the diagnosis and treatment information conforms to the clinical pathway participation criteria, for the purpose of determining whether a clue about medical adverse events exists in the diagnosis and treatment information, as an embodiment, it is possible to identify the diagnosis and treatment information format category to which the diagnosis and treatment information belongs, and then select, according to the diagnosis and treatment information format category, the knowledge base of clinical pathway medical orders or the keyword library of medical adverse events as the basis for determining whether a clue about medical adverse events exists in the diagnosis and treatment information.

During the above implementation process, the knowledge base of clinical pathway medical orders or the keyword library of medical adverse events is selected according to the diagnosis and treatment information format category, so as to determine whether a clue about medical adverse events exists in the diagnosis and treatment information; in this way, it is possible to perform a comprehensive review of the diagnosis and treatment information, so as to discover an abnormal medical action of a doctor during the diagnosis and treatment process in time, hereby monitoring medical adverse events during the diagnosis and treatment process of patients.

Here, the diagnosis and treatment information format category may contain medical orders and medical documents, that is to say, when recording the diagnosis and treatment information, the medical care personnel may make records in accordance with the format of medical orders or in accordance with the format of medical documents. For each diagnosis and treatment information format category, a different way may be selected to identify whether a clue about medical adverse events exists in the diagnosis and treatment information. As an embodiment, in case that the diagnosis and treatment information format category concerns medical documents, the diagnosis and treatment information is matched with respective preset keywords in the keyword library of medical adverse events; and if a keyword matching the respective preset keywords exists in the diagnosis and treatment information, it is determined that a clue about medical adverse events exists in the diagnosis and treatment information.

During the above implementation process, by performing keyword matching on the diagnosis and treatment information, it is possible to quickly and accurately identify whether a clue about medical adverse events exists in the diagnosis and treatment information.

The keyword library of medical adverse events is pre-built for clues about medical adverse events, it involves words which may result in clues about medical adverse events during processes of such as diagnosis and treatment procedure, disease symptoms, relevant diagnosis, and the keyword library is able to reflect medical scenes and service contents such as hospitalization, operation, and nursing. In case that the medical care personnel misconduct themselves during the diagnosis and treatment, relevant sensitive words would be recorded in the hospital system in the form of medical documents. The keyword library may be divided into levels 1 to 3 according to the classification hierarchy, wherein level 1 category has the widest coverage and is divided into nursing, medical instrument, operation, blood transfusion and endoscopy; level 2 category has a smaller coverage; and level 3 category has the smallest coverage but with better refinement, which may be more easily matched accordingly, and an example of the keyword library is shown in Table 2.

TABLE 2

| level 1 category | level 2 category | level 3 category |
|---|---|---|
| adverse events | adverse patient | Bedsore |
| associated with | management | fall\falling down from bed\scald |
| nursing | | aspiration\asphyxia |
| | | lost\fight\suicide |
| | | refusal of infusion\refusal of |
| | | intubation\refusal of blood |
| | | transfusion |
| | medication errors | no skin test\positive skin test |
| | | medication + anaphylaxis |
| | | no skin test\positive skin test |
| | | medication + anaphylactic shock |
| | | no skin test\positive skin test |
| | | medication + patient death |
| | | drug poisoning |
| | specimen | specimen is not collected and |
| | collection errors | transported in time |
| | | specimen is missing |
| | | specimens of urine\sputum\pleural |
| | | effusion\peritoneal effusion\ |
| | | cerebrospinal fluid\articular cavity |
| | | fluid are too little |
| | | coagulation of blood specimen |
| | | formation of subcutaneous |
| | | hematoma after specimen collection |
| | infusion-related | infusion + formation of subcutaneous |
| | adverse events | hematoma |
| | | infusion seepage |
| | | infusion + chill\fever\rigor |
| | | infusion + phlebitis |
| | | infusion + air embolism |
| | | infusion + heart failure\pulmonary |
| | | edema |
| | | infusion + shock |
| | | infusion + bacteremia\infusion- |
| | | related septicemia |
| | | infusion + rash\infusion-related |
| | | dyspnea\infusion-related |
| | | disturbance of consciousness\ |
| | | infusion-related angioedema |
| | catheter-related | self-extubation of drainage tube/ |
| | adverse events | self-extubation of urethral catheter |
| | | drainage tube slippage\urethral |
| | | catheter slippage |
| | | bile leakage\leakage of intestinal |
| | | fluid\leakage of liver ascites\ |
| | | leakage of gastric fluid\leakage of |
| | | pleural effusion |
| adverse events | retention of | surgical instruments\blade\suture |
| associated with | medical supplies | needle\needle breakage |
| medical | | dressing retention\gauze retention\ |
| instrument | | cotton ball retention\non-absorbable |
| | | surgical suture retention |
| | | drainage tube fragment retention\ |
| | | catheter fragment retention |
| | improper use of | infection\suppuration\oozing of |
| | medical | blood at the puncture site of a |
| | instrument | disposable infusion set; |
| | | infection\suppuration\oozing of |
| | | blood at the puncture site of a |
| | | remaining needle; |
| | | infection\suppuration\oozing of |
| | | blood at the puncture site of a |
| | | disposable sterilized syringe; |
| | | infection\suppuration\oozing of |
| | | blood at the puncture site of an |
| | | insulin pump; |
| | | infection\suppuration\oozing of |
| | | blood at the puncture site of a |
| | | non-absorbable suture |
| | | pneumothorax after thoracentesis\ |
| | | hemothorax after thoracentesis |
| | | hemorrhage after bone marrow |
| | | aspiration\hemorrhage after bone |
| | | marrow biopsy |
| | | hemodialysis + bacteremia\ |
| | | hemodialysis + septicemia; |
| | | hemodialysis + HIV infection\ |
| | | hemodialysis + AIDS; |
| | | hemodialysis + HBV infection\ |
| | | hemodialysis + hepatitis B; |
| | | hemodialysis + TP infection\ |
| | | hemodialysis + syphilis; |
| | | hemodialysis + HCV infection\ |
| | | hemodialysis + hepatitis c |
| | | ventilator-associated bronchitis\ |
| | | ventilator-associated pneumonia\ |
| | | ventilator-associated |
| | | bronchopneumonia |
| | | breakage of bone plate\bending of |
| | | bone plate\looseness of bone plate\ |
| | | rejection reaction of bone plate\ |
| | | allodynia at the bone fracture site\ |
| | | nonunion of fracture at the bone |
| | | fracture site\infection at the bone |
| | | fracture site; |
| | | looseness of bone needle\rejection |
| | | reaction of bone needle |
| | | slow absorption of an absorbable |
| | | suture\rejection reaction at |
| | | absorbable suture wound\infection |
| | | at absorbable suture wound |
| | | urinary tract infection triggered by |
| | | urethral catheter\acute purulent |
| | | cholangitis triggered by drainage |
| | | tube\secondary purulent peritonitis |
| | | triggered by drainage tube\acute |
| | | empyema triggered by drainage tube |
| adverse events | intraoperative | intraoperation + hemorrhage |
| associated with | adverse events | intraoperation + burn\scald |
| operations | | intraoperation + irreparable injury to |
| | | heart\liver\bile duct\spleen\lung\ |
| | | kidney\pancreas\intestinal tract\ |
| | | blood vessel\muscle\nerve |
| | | ischemia-reperfusion injury to |
| | | transplanted kidney\liver\heart\lung |
| | postoperative | postoperation + wound inflammation\ |
| | adverse events | postoperation + wound purulence\ |
| | | postoperation + wound dehiscence\ |
| | | postoperation + delayed wound |
| | | healing\postoperation + non-healing |
| | | wound |
| | | operative site infection |
| | | postoperation + septicemia\ |
| | | postoperation + bacteremia\ |
| | | postoperation + pneumonia\ |
| | | postoperation + systemic |
| | | inflammatory response syndrome |
| | | postoperation + intestinal tract |
| | | adhesion\postoperation + peritonitis |
| | | postoperation + pulmonary embolism\ |
| | | postoperation + air embolism\ |
| | | postoperation + venous embolism |
| | | postoperation + sinus tract formation |
| | | postoperation + hemorrhage\ |
| | | postoperation + hemorrhagic shock |
| | | postoperation + dyspnea\ |
| | | postoperation + persistent hyoxemia |
| | | postoperation + hepatic failure\ |
| | | postoperation + renal failure\ |
| | | postoperation + multiorgan |
| | | disfunction syndrome |
| | | postoperation + arrhythmia\ |
| | | postoperation + acute coronary |
| | | syndrome\postoperation + sudden |
| | | cardiac arrest\postoperation + heart |
| | | failure |
| | anesthesia-related | The operation is cancelled, after the |
| | adverse events | anesthesia begins, but before the |
| | | operation begins |
| | | hoarseness after endotracheal |
| | | intubation under general anesthesia |
| | | death within 24 hours after the begin |
| | | of anesthesia |
| | | postoperative premature extubation + |
| | | vegetative state of a patient |
| | | newly occurred coma after |
| | | anesthesia |
| adverse events | acute hemolytic | blood transfusion + acute renal |
| associated with | transfusion | failure |
| blood | reactions | blood transfusion + systemic |
| transfusion | | inflammatory response syndrome |
| | | blood transfusion + shock triggered |
| | | by hemolytic anemia |
| | | blood transfusion + disseminated |
| | | intravascular coagulation |
| | non-hemolytic | blood transfusion + graft-versus-host |
| | transfusion | disease |
| | reactions | blood transfusion + bacteremia\ |
| | | blood transfusion + septicemia; |
| | | blood transfusion + HIV infection\ |
| | | blood transfusion + AIDS; |
| | | blood transfusion + HBV infection\ |
| | | blood transfusion + hepatitis B; |
| | | blood transfusion + TP infection\ |
| | | blood transfusion + syphilis; |
| | | blood transfusion + HCV infection\ |
| | | blood transfusion + hepatitis c |
| | | blood transfusion + congestive heart |
| | | failure/pulmonary edema |
| adverse events | adverse events | hemorrhage after gastric endoscopy\ |
| associated with | associated with | hemorrhage after intestinal |
| endoscopy | gastrointestinal | endoscopy; |
| | endoscopy | hemorrhagic shock after gastric |
| | | endoscopy\hemorrhagic shock after |
| | | intestinal endoscopy |
| | adverse events | complicated pneumothorax after |
| | associated with | mediastinoscopy; |
| | respiratory | hemorrhage after mediastinoscopy |
| | endoscopy | complicated respiratory tract |
| | | infection after bronchoscopy; |
| | | complicated hemorrhage after |
| | | bronchoscopy; |
| | | complicated hyoxemia after |
| | | bronchoscopy; |
| | | complicated sudden cardiac arrest |
| | | after bronchoscopy |
| | | complicated pneumothorax after |
| | | thoracoscopy; |
| | | complicated hemothorax after |
| | | thoracoscopy; |
| | | complicated infection after |
| | | thoracoscopy |
| | adverse events | pneumoperitoneum after |
| | associated with | laparoscopy\abdominal |
| | laparoscopy | compartment syndrome after |
| | | laparoscopy |
| | | intestinal injury after laparoscopy |
| | | hemorrhage at the puncture site after |
| | | laparoscopy |

Thus, a search can be made in the keyword library of medical adverse events to find out whether the diagnosis and treatment information contains a preset keyword in the level 3 category in Table 2 above, wherein if yes, this keyword can be extracted, and the level 2 category and/or the level 1 category, to which this keyword belongs, can be deduced, in this way, a clue about medical adverse event can be generated, hereby finding out the cause for the generation of the clue about medical adverse events.

Of course, if the diagnosis and treatment information format category, to which the diagnosis and treatment information belongs, concerns medical documents, it can also be searched according to the above mode to find out whether a preset keyword exists in the diagnosis and treatment information, wherein if yes, it indicates that there is a clue about medical adverse events in the diagnosis and treatment information.

However, if the diagnosis and treatment pathway fails to match the clinical pathway participation criteria, and the diagnosis and treatment information format category, to which the diagnosis and treatment information belongs, concerns medical orders, the medical orders thereof cannot be identified to find out whether a clue about medical adverse events exists, as it is not admitted to the clinical pathways yet; at this moment, the procedure can be ended directly, and it is identified automatically that there is no clue about medical adverse events in the diagnosis and treatment information.

During the above implementation process, by performing keyword matching on the diagnosis and treatment information, it is possible to quickly and accurately identify whether a clue about medical adverse events exists in the diagnosis and treatment information.

As an embodiment, in case that the diagnosis and treatment information format category concerns medical orders, medical order information in the diagnosis and treatment information is matched with the corresponding knowledge base of clinical pathway medical orders, and if in the medical order information, there is medical order information not belonging to the knowledge base of clinical pathway medical orders, it is determined that there is a clue about medical adverse events in the diagnosis and treatment information.

Doctors can implement diagnosis and treatment actions by issuing medical orders, a clue about medical adverse events can be dug out accordingly by capturing medical order information in the diagnosis and treatment information, and it is helpful to normalize diagnosis and treatment actions of doctors by constructing a knowledge base of clinical pathway medical orders. The knowledge base of clinical pathway medical orders is structurally constructed according to the medical order information. According to the time limit of medical orders, they are divided into standing orders and stat orders; according to the application scenes of medical orders, they are divided into medicine-related orders, nursing-related orders, test-related orders and the like. The structural construction is conducive to clue digging and refined information management.

Here, an example of the knowledge base of clinical pathway medical orders is shown in Table 3 as follows.

TABLE 3

| | required medical orders | | optional medical orders | |
|---|---|---|---|---|
| Standing | nursing- | order 1 | nursing- | order 1 |
| orders | related | . . . | related | . . . |
| | orders | order n | orders | order n |
| | medicine- | order 1 | medicine- | order 1 |
| | related | . . . | related | . . . |
| | orders | order n | orders | order n |
| | test- | order 1 | test- | order 1 |
| | related | . . . | related | . . . |
| | orders | order n | orders | order n |
| | . . . | | | |
| Stat | nursing- | order 1 | nursing- | order 1 |
| orders | related | . . . | related | . . . |
| | orders | order n | orders | order n |
| | medicine- | order 1 | medicine- | order 1 |
| | related | . . . | related | . . . |
| | orders | order n | orders | order n |
| | test- | order 1 | test- | order 1 |
| | related | . . . | related | . . . |
| | orders | order n | orders | order n |

. . .

Thus, if in the medical order information of the diagnosis and treatment information, there is medical order information not belonging to the knowledge base of clinical pathway medical orders, it is indicated that new medical orders are possibly added into the diagnosis and treatment information thereof, and a clue about medical adverse events exists; and if the medical order information in the diagnosis and treatment information belongs to the medical order information in the knowledge base of clinical pathway medical orders, the absence of a clue about medical adverse events is indicated.

During the above implementation process, by identifying the medical order information in the diagnosis and treatment information, it is possible to quickly and accurately identify whether a clue about medical adverse events exists in the diagnosis and treatment information.

In order to prevent potential risks of medical safety caused by covering up clues about medical adverse events, after determining that there is an adverse event in the diagnosis and treatment information as described above, a medical adverse even clue report may further be generated on the basis of the clue about medical adverse events, which is then sent to the user terminal of medical care quality management personnel.

During the above implementation process, the medical care quality management personnel are enabled to manage and control abnormal medical actions by sending the medical adverse event clue report generated from the clue about medical adverse events to the medical care quality management personnel.

For example, a medical adverse event clue report can be formed with a clue of “pathway exceeded-standing\stat order-medical order category-medical order item”, when the presence of a clue about medical adverse events is determined by matching the knowledge base of clinical pathway medical orders as described above. For medical documents, information about abnormal complications is captured by matching the keyword library of medical adverse events, and a report with a clue of “combined abnormal complications-keyword” is formed.

Here, the medical care quality management personnel may refer to the medical care quality management committee that can view clues about medical adverse events through the user terminal, so as to completely eradicate the occurrence of omission and concealment of clues about medical adverse events and to improve the medical care quality management capability of the whole hospital. The medical adverse event clue report may be in forms as shown in Table 4 and Table 5 as follows.

TABLE 4
Medical Adverse Event Clue Report

| | | | | | |
|---|---|---|---|---|---|
| Department | xxx | Person | xxx | Submission | xxx |
| | | in | | time | |
| | | charge | | | |

| General patient information | | | | Clues about medical adverse events | |
|---|---|---|---|---|---|
| Name | xxx | Gender | xxx | Clue 1 | Level 1 category- level 2 |
| | | | | | category- level 3 category |
| Age | xxx | Patient | xxx | . . . | . . . |
| | | ID | | | |

TABLE 5
Medical Adverse Event Clue Report

| Department | xxx | Person | xxx | | Submission | xxx |
|---|---|---|---|---|---|---|
| | | in | | | time | |
| | | charge | | | | |
| General patient information | | | | Clues about medical adverse events | | |
| Name | xxx | Gender | xxx | Clue 1 | Pathway exceeded-standing order- | |
| | | | | | medicine related-medicine name | |
| Age | xxx | Patient | xxx | . . . | . . . | |
| | | ID | | | | |

On the basis of this medical adverse event clue report, the patient ID can be acquired therefrom, and all relevant clues about medical adverse events of the patient during the hospitalization can then be inquired, which would be helpful for the medical care quality management personnel to investigate the situations of clues about medical adverse events of the patient, and a medical adverse event clue library is formed, which is shown in Table 6 as follows.

TABLE 6

| General patient information | Clues about medical adverse events | | |
|---|---|---|---|
| Name xx | Clue 1 | . . . | Clue n |
| Gender xx | Department xxx | . . . | Department xxx |
| Age xx | Person in charge xxx | | Person in charge xxx |
| Patent ID xxx | Submission time xxx | | Submission time xxx |
| | Clue information xxx | | Clue information xxx |
| | Verification result xxx | | Verification result xxx |

In this way, according to the patient ID, the medical care quality management personnel can inquire about all relevant clue information of the patient during the hospitalization in the system, browse through the patient-centered medical adverse event clue library, and can inquire based on the medical adverse event clue report of the patient about abnormal information of specific diagnosis and treatment actions involved in the report, such that the medical care quality management personnel can manage and control abnormal medical actions.

In addition, the medical adverse event clue report can further be sent to the user terminal of the medical care personnel causing the clue.

During the above implementation process, the medical care personnel are enabled to further verify the medical adverse event by sending the medical adverse event clue report generated from the clue about medical adverse events to the medical care personnel.

The medical adverse event clue report can further contain the ID of the medical care personnel causing the clue about medical adverse events, thus, the medical adverse event clue report can also be sent to the user terminal of the medical care personnel, and the medical care personnel can discriminate the clue about medical adverse events after obtaining the medical adverse event clue report. The option “Yes” or “No” regarding the clue about medical adverse events can then be selected in the system, wherein if “Yes” is selected, the event is reported by directly invoking the medical adverse event reporting system; and if “No” is selected, the system would automatically send this medical adverse event clue report to the corresponding user terminal of the medical care quality management personnel for further verification.

When making verification, the medical care quality management personnel can obtain the 360 view of the electronic medical record of the patent according to the general patient information, browse through the holographic diagnosis and treatment data, including the historical admission diagnosis and treatment data of the patient, and perform multi-dimensional visual analysis in aspects of time, disease entities, test and examination items and the like, which helps the medical care quality management personnel with quick grasping of the diagnosis and treatment situation of the patient and facilitates the accurate verification of the medical adverse events. If the medical care quality management personnel confirm after verification that this clue about medical adverse events originates from a real medical adverse event, the result can be immediately fed through the system to relevant person in charge, who is then prompted to report the result in time.

The above implementation process is introduced below through specific examples.

Example 1: Female patient aged 83. Diagnosis: right femoral neck fracture, pulmonary heart disease, brain atrophy, severe, first-level nursing. The patient suffered from right hip pain after trauma, was limited in movement for 5 hours, and was admitted to orthopedics department. The patient was sent to the ward using a gurney, with clear consciousness, taking the semi-recumbent position, the affected limb being raised and braking being taken. 3 days later, by morning care shift change, it is found that the bed and the hip skin were contaminated by urination and defecation of the patient; her family member offered the use of adult diapers, and the skin was cleaned immediately, and the bed sheet was changed; and urinary catheterization was maintained, the pressurized part was protected by skin bandages, and the turnover of the body was strictly conducted. After 10 days in the hospital, it is found by the shift change in the morning that there was a large area of scattered reddish blisters on the right hip and pressure sores were formed, which was faithfully described in the shift record.

General patient information was formed in the hospitalization system of the hospital through the admission registration process of the patient, including patient ID, name, gender, age and the like. After the admission of the patient, admission record was formed and submitted by doctors. According to the submitted information, the system identified the diagnosis and treatment information as “right femoral fracture, pulmonary heart disease”. By matching the diagnosis and treatment information with the clinical pathway participation criteria, there was no perfect match in the system, and it was identified that the information submitted in the system relates to medical documents, accordingly, a keyword library of medical adverse events matching was performed in the system. If the presence of a preset keyword was not found, the subsequent diagnosis and treatment information determination would be continued. Subsequently, doctors issued medicine-related and test- and examination-related medical orders and submitted the same, the system identified that they were not medical documents, and subsequent keyword library of medical adverse events matching would not be performed. Nurses followed the medical orders for nursing actions. After the shift change, nurses generated and submitted shift records. According to the submitted information, the system identified the same as medical documents, and a keyword library of medical adverse events matching was performed. By matching the keyword library of medical adverse events, the keyword “pressure sore” was found in the system. A medical adverse event clue report as shown in Table 7 below was automatically formed in the system and a reminder was sent to the nurse LI Si, and an option of asking whether it is identified as a clue about medical adverse events popped up.

TABLE 7
Medical Adverse Event Clue Report

| | | | | | |
|---|---|---|---|---|---|
| Department | Orthopedics | Person | LI Si | Submission | 2019.12.12.10:05:35 |
| | department | in | | time | |
| | | charge | | | |

| General patient information | | | | Clues about medical adverse events |
|---|---|---|---|---|
| Name | xx | Gender | female | Clue 1 |
| Age | 83 | Patient | xx | Adverse events associated with |
| | | ID | | nursing-adverse patient |
| | | | | management-pressure sore |

LI Si believed that this event is unqualified to be reported to the medical adverse event system, and selected the option “No”. The report was further forwarded to the user terminal of the medical care quality management personnel. According to the patient information in the report, the medical care quality management personnel inquired about other diagnosis and treatment information of the patient in the 360 view of the electronic medical record of the patient, and it is found out that the patient was aged, suffered from severe illness of right femoral neck fracture complicated by pulmonary heart disease, which results in a forced posture, and it can be deemed as inevitable bed sores and is thus not identified as a clue about medical adverse events. An option of asking whether it is identified as a clue about medical adverse events popped up in the system. The medical care quality management personnel selected the option “No”, and the procedure of this event ended.

Example 2: Female patient aged 47 was diagnosed as having osteoarthritis and was admitted to the department of bones and joints; after 5 days, she underwent bilateral total downside knee arthroplasty of general anesthesia, and was sent back to the joint ward after postoperative extubation. But she suffered sudden difficulty in breathing when just being sent back to the ward, and after the emergency treatment, failed to regain the consciousness and was in the vegetative state. Two months after the operation, she was transferred to another hospital for hyperbaric oxygen therapy, but the effect was not obvious. Two and a half months after the operation, the patient was continuously treated in a local hospital.

General patient information was formed in the hospitalization system of the hospital through the admission registration process of the patient, including patient ID, name, gender, age and the like. After the admission of the patient, admission record was formed and submitted by doctors. According to the submitted information, the system identified the disease diagnosis and treatment information “osteoarthritis” and identified the operation information “knee arthroplasty”. By matching the diagnosis and treatment information with the clinical pathway participation criteria, there is a perfect match in the system. The submitted information was identified by the system as medical documents, and a keyword library of medical adverse events matching was performed once, wherein no clue was found. Doctors issued medicine-related and test- and examination-related medical orders and submitted the same, the system identified that the submitted information was medical orders, and no clue was found during the knowledge base of clinical pathway medical orders matching. The patient underwent knee arthroplasty, and was sent back to the ward after postoperative extubation. But she suffered sudden difficulty in breathing and lost the consciousness, doctors immediately performed emergency treatment, issued medical orders and submitted the same. The system identified the submitted information as medical orders, and performed medical order knowledge base matching, wherein newly added medical order contents were found, which exceeded the knowledge base of clinical pathway medical orders, a medical adverse event clue report as shown in Table 8 below was formed, a reminder was sent to the doctor YAO Wu, and an option of asking whether it is identified as a clue about medical adverse events popped up.

TABLE 8
Medical Adverse Event Clue Report

| | | | | | |
|---|---|---|---|---|---|
| Department | Orthopedics | Person | YAO | Submission | 2019.12.12.10:05:36 |
| | department | in | Wu | time | |
| | | charge | | | |

| General patient information | | | | Clues about medical adverse events |
|---|---|---|---|---|
| Name | xx | Gender | female | Clue 1 |
| Age | 47 | Patient | xx | Pathway exceeded-stat |
| | | ID | | order-medicine related-epinephrine |
| | | | | Clue 2 |
| | | | | Pathway exceeded-stat |
| | | | | order-medicine related-coramine |
| | | | | Clue 3 |
| | | | | Pathway exceeded-stat |
| | | | | order-treatment related- |
| | | | | cardio-pulmonary resuscitation |

YAO Wu deliberately concealed clues about medical adverse events, and selected the option “No” so as to avoid reporting the event. According to the patient information in the report, the medical care quality management personnel inquired about other diagnosis and treatment information of the patient in the 360 view of the electronic medical record of the patient, and it is found out that the patient suffered from osteoarthritis, which is not complicated by other severe illnesses, thus, the need for emergency treatment after knee arthroplasty is unreasonable and is accordingly deemed as a clue about medical adverse events. An option of asking whether it is identified as a clue about medical adverse events popped up in the system, and the medical care quality management personnel selected the option “Yes”, an event feedback report was accordingly sent to YAO Wu through the system, who was then prompted to report this medical adverse event, and the procedure ended.

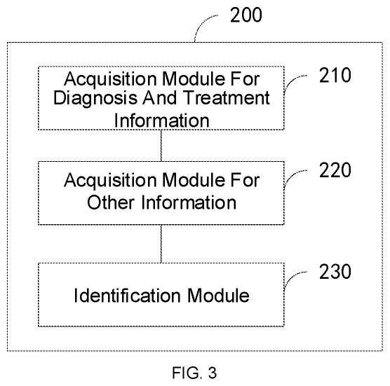

Referring to FIG. 3, FIG. 3 is a structure diagram of a device 200 for identifying clues about medical adverse events provided in an embodiment of the present disclosure, this device 200 may be a module, a program segment or a code on an electronic equipment. It shall be understood that this device 200 corresponds to the method embodiment of FIG. 2 above, and can carry out respective steps involved in the method embodiment of FIG. 2; as for the specific functions of the device 200, reference can be made to the preceding description, and in order to avoid repetition, detailed description is properly omitted here.

Optionally, the device 200 comprises:

an acquisition module for diagnosis and treatment information 210 configured to acquire diagnosis and treatment information on a patient;

an acquisition module for other information 220 configured to acquire a knowledge base of clinical pathway medical orders or a keyword library of medical adverse events corresponding to the diagnosis and treatment information; and

an identification module 230 configured to determine whether a clue about medical adverse events exists in the diagnosis and treatment information based on the knowledge base of clinical pathway medical orders or the keyword library of medical adverse events.

Optionally, the identification module 230 is configured to identify the diagnosis and treatment information format category to which the diagnosis and treatment information belongs; and select, according to the diagnosis and treatment information format category, the knowledge base of clinical pathway medical orders or the keyword library of medical adverse events as the basis for determining whether a clue about medical adverse events exists in the diagnosis and treatment information.

Optionally, the identification module 230 is configured to match the diagnosis and treatment information with respective preset keywords in the keyword library of medical adverse events, in case that the diagnosis and treatment information format category concerns medical documents; and determine that there is a clue about medical adverse events in the diagnosis and treatment information, if there is a keyword in the diagnosis and treatment information matching the respective preset keywords.

Optionally, the identification module 230 is configured to match medical order information in the diagnosis and treatment information with the knowledge base of clinical pathway medical orders, in case that the diagnosis and treatment information format category concerns medical orders; and determine that there is a clue about medical adverse events in the diagnosis and treatment information, if in the medical order information, there is medical order information not belonging to the knowledge base of clinical pathway medical orders.

Optionally, the device 200 further comprises:

a first sending module configured to generate a medical adverse event clue report based on the clue about medical adverse events; and send the medical adverse event clue report to the user terminal of medical care quality management personnel.

Optionally, the device 200 further comprises:

a second sending module configured to generate a medical adverse event clue report based on the clue about medical adverse events; and send the medical adverse event clue report to the user terminal of the medical care personnel causing the clue about medical adverse events.

An embodiment of the present disclosure provides a memory medium, wherein the method process implemented by the electronic equipment in the method embodiment as shown in FIG. 2 is carried out, when the computer program is executed by the processor.

The present embodiment discloses a computer program product, comprising a computer program stored in a non-transient computer memory medium, with the computer program comprising a programmed instruction, wherein when the programmed instruction is executed by a computer, the computer can implement the method provided in the respective method embodiments above, comprising: e.g. acquiring diagnosis and treatment information on a patient; acquiring a knowledge base of clinical pathway medical orders or a keyword library of medical adverse events corresponding to the diagnosis and treatment information; and determining whether a clue about medical adverse events exists in the diagnosis and treatment information based on the knowledge base of clinical pathway medical orders or the keyword library of medical adverse events.

In summary, a method and a device for identifying clues about medical adverse events, an electronic equipment, and a memory medium are provided in the embodiments of the present disclosure, wherein clues about medical adverse events can be dug out initiatively by determining whether a clue about medical adverse events exists in the diagnosis and treatment information based on the knowledge base of clinical pathway medical orders or the keyword library of medical adverse events, which reduces manual intervention in the identification of medical adverse events, lowers manpower consumption, and improves work efficiency.

In the embodiments provided in the present disclosure, it shall be understood that the disclosed device and method may be implemented in other ways. The embodiments of device described above are merely schematic: for example, the unit division refers to merely a division of logical functions, and during practical implementation, it may be divided in other ways; for example, a plurality of units or assemblies may be combined or integrated into another system, or some features may be ignored or may not be implemented. Further, the displayed or discussed mutual coupling or direct coupling or communication connection may be indirect coupling or communication connection via certain communication interfaces, devices or units, and may be electrical, mechanical coupling or communication connection, or coupling or communication connection in other forms.

In addition, units described as separate components may be or may not be physically separate, components displayed as units may be or may not be physical units, i.e. may be placed at one location, or may be distributed on a plurality of network elements. Partial or all units may be selected according to actual requirements to achieve the purpose of the solution of the present embodiment.

Moreover, individual functional modules in the individual embodiments of the present disclosure may be integrated together to form an independent part, or individual modules may exist separately, or two or more modules may be integrated to form an independent part.

In the context, relational terms such as first and second or the like are used only to distinguish one entity or operation from another entity or operation, and do not necessarily require or imply that there is any such practical relation or sequence among these entities or operations.

The above mentioned is merely embodiments of the present disclosure, and is not intended to limit the scope of protection of the present disclosure, and for a person skilled in the art, the present disclosure may be modified and changed in various ways. Any modifications, equivalent substitutions and improvements made within the spirit and the principle of the present disclosure shall all be covered in the scope of protection of the present disclosure.

1. A method for identifying clues about medical adverse events, comprising following steps of:
acquiring diagnosis and treatment information on a patient;
acquiring a knowledge base of clinical pathway medical orders or a keyword library of medical adverse events which is corresponding to the diagnosis and treatment information; and
determining, based on the knowledge base of clinical pathway medical orders or the keyword library of medical adverse events, whether a clue about medical adverse events exists in the diagnosis and treatment information.

2. The method according to claim 1, wherein the step of determining whether a clue about medical adverse events exists in the diagnosis and treatment information based on the knowledge base of clinical pathway medical orders or the keyword library of medical adverse events comprises following steps of:
identifying a diagnosis and treatment information format category to which the diagnosis and treatment information belongs; and
selecting, according to the diagnosis and treatment information format category, the knowledge base of clinical pathway medical orders or the keyword library of medical adverse events as a basis for determining whether a clue about medical adverse events exists in the diagnosis and treatment information.

3. The method according to claim 2, wherein the step of selecting, according to the diagnosis and treatment information format category, the knowledge base of clinical pathway medical orders or the keyword library of medical adverse events as the basis for determining whether a clue about medical adverse events exists in the diagnosis and treatment information comprises following steps of:
matching the diagnosis and treatment information with respective preset keywords in the keyword library of medical adverse events, in case that the diagnosis and treatment information format category concerns medical documents; and
determining that a clue about medical adverse events exists in the diagnosis and treatment information if a keyword matching the respective preset keyword exists in the diagnosis and treatment information.

4. The method according to claim 2, wherein the step of selecting, according to the diagnosis and treatment information format category, the knowledge base of clinical pathway medical orders or the keyword library of medical adverse events as the basis for determining whether a clue about medical adverse events exists in the diagnosis and treatment information comprises following steps of:
matching medical order information in the diagnosis and treatment information with the knowledge base of clinical pathway medical orders, in case that the diagnosis and treatment information format category is of medical orders; and
determining that a clue about medical adverse events exists in the diagnosis and treatment information, if medical order information which does not belong to the knowledge base of clinical pathway medical orders exists in the medical order information.

5. The method according to claim 1, wherein after determining that a clue about medical adverse events exists in the diagnosis and treatment information, the method further comprises following steps of:
generating a medical adverse event clue report based on the clue about medical adverse events; and
sending the medical adverse event clue report to a user terminal of medical care quality management personnel.

6. The method according to claim 1, wherein after determining that a clue about medical adverse events exists in the diagnosis and treatment information, the method further comprises following steps of:
generating a medical adverse event clue report based on the clue about medical adverse events; and
sending the medical adverse event clue report to a user terminal of the medical care personnel causing the clue.

7. A device for identifying a clue about medical adverse events, comprising:
an acquisition module for diagnosis and treatment information configured to acquire diagnosis and treatment information on a patient;
an acquisition module for other information configured to acquire a knowledge base of clinical pathway medical orders or a keyword library of medical adverse events which is corresponding to the diagnosis and treatment information; and
an identification module configured to determine whether a clue about medical adverse events exists in the diagnosis and treatment information based on the knowledge base of clinical pathway medical orders or the keyword library of medical adverse events.

8. The device according to claim 7, wherein the identification module is configured to identify a diagnosis and treatment information format category to which the diagnosis and treatment information belongs; and select, according to the diagnosis and treatment information format category, the knowledge base of clinical pathway medical orders or the keyword library of medical adverse events as a basis for determining whether a clue about medical adverse events exists in the diagnosis and treatment information.

9. An electronic equipment, comprising a processor and a memory in which computer readable instructions are stored, wherein a method according to claim 1 is implemented when the computer readable instructions are executed by the processor.

10. The method according to claim 2, wherein after determining that a clue about medical adverse events exists in the diagnosis and treatment information, the method further comprises following steps of:
generating a medical adverse event clue report based on the clue about medical adverse events; and
sending the medical adverse event clue report to a user terminal of medical care quality management personnel.

11. The method according to claim 3, wherein after determining that a clue about medical adverse events exists in the diagnosis and treatment information, the method further comprises following steps of:
generating a medical adverse event clue report based on the clue about medical adverse events; and
sending the medical adverse event clue report to a user terminal of medical care quality management personnel.

12. The method according to claim 4, wherein after determining that a clue about medical adverse events exists in the diagnosis and treatment information, the method further comprises following steps of:
generating a medical adverse event clue report based on the clue about medical adverse events; and
sending the medical adverse event clue report to a user terminal of medical care quality management personnel.

13. The method according to claim 2, wherein after determining that a clue about medical adverse events exists in the diagnosis and treatment information, the method further comprises following steps of:
generating a medical adverse event clue report based on the clue about medical adverse events; and
sending the medical adverse event clue report to a user terminal of the medical care personnel causing the clue.

14. The method according to claim 3, wherein after determining that a clue about medical adverse events exists in the diagnosis and treatment information, the method further comprises following steps of:
generating a medical adverse event clue report based on the clue about medical adverse events; and
sending the medical adverse event clue report to a user terminal of the medical care personnel causing the clue.

15. The method according to claim 4, wherein after determining that a clue about medical adverse events exists in the diagnosis and treatment information, the method further comprises following steps of:
generating a medical adverse event clue report based on the clue about medical adverse events; and
sending the medical adverse event clue report to a user terminal of the medical care personnel causing the clue.

16. The electronic equipment according to claim 9, wherein when the computer readable instructions are executed by the processor, the processor is configured to execute the following:
identifying a diagnosis and treatment information format category to which the diagnosis and treatment information belongs; and
selecting, according to the diagnosis and treatment information format category, the knowledge base of clinical pathway medical orders or the keyword library of medical adverse events as a basis for determining whether a clue about medical adverse events exists in the diagnosis and treatment information.

17. The electronic equipment according to claim 16, wherein when the computer readable instructions are executed by the processor, the processor is configured to execute the following:
matching the diagnosis and treatment information with respective preset keywords in the keyword library of medical adverse events, in case that the diagnosis and treatment information format category is of medical documents; and
determining that a clue about medical adverse events exists in the diagnosis and treatment information if a keyword matching the respective preset keyword exists in the diagnosis and treatment information.

18. The electronic equipment according to claim 16, wherein when the computer readable instructions are executed by the processor, the processor is configured to execute the following:
matching medical order information in the diagnosis and treatment information with the knowledge base of clinical pathway medical orders, in case that the diagnosis and treatment information format category is of medical orders; and
determining that a clue about medical adverse events exists in the diagnosis and treatment information, if medical order information which does not belong to the knowledge base of clinical pathway medical orders exists in the medical order information.

19. The electronic equipment according to claim 9, wherein when the computer readable instructions are executed by the processor, the processor is configured to execute the following:
generating a medical adverse event clue report based on the clue about medical adverse events; and
sending the medical adverse event clue report to a user terminal of medical care quality management personnel.

20. The electronic equipment according to claim 9, wherein when the computer readable instructions are executed by the processor, the processor is configured to execute the following:
generating a medical adverse event clue report based on the clue about medical adverse events; and
sending the medical adverse event clue report to a user terminal of the medical care personnel causing the clue.
